SEQUENCE LISTING (16 sequences)

SEQ ID NO: 1 - protein, 21 aa, Artificial Sequence
  Modified Sequence
MSRIVINAYAKATQPDFKSHM

SEQ ID NO: 2 - protein, 35 aa, Artificial Sequence
  Modified Sequence
MEKHTKDQIIEAPHMSRIVINAYAKATQPDFKSHM

SEQ ID NO: 3 - protein, 94 aa, Artificial Sequence
  Modified Sequence
MGSSHHHHHHSSGLVPRGSHMSRIVINAYAKATQPDFKSHMFVNQHLCGSHLVEALYLVCGERGFFYTPKTKRGIVEQCCTSICSLYQLENYCN

SEQ ID NO: 4 - protein, 88 aa, Artificial Sequence
  Modified Sequence
MEKHTKDQIIEAPHMSRIVINAYAKATQPDFKSHMFVNQHLCGSHLVEALYLVCGERGFFYTPKTKRGIVEQCCTSICSLYQLENYCN

SEQ ID NO: 5 - protein, 94 aa, Artificial Sequence
  Modified Sequence
MGSSHHHHHHSSGLVPRGSHMSRIVINAYAKATQPDFKSHMFVNQHLCGSHLVEALYLVCGERGFFYTKPTKRGIVEQCCTSICSLYQLENYCN

SEQ ID NO: 6 - protein, 88 aa, Artificial Sequence
  Modified Sequence
MEKHTKDQIIEAPHMSRIVINAYAKATQPDFKSHMFVNQHLCGSHLVEALYLVCGERGFFYTKPTKRGIVEQCCTSICSLYQLENYCN

SEQ ID NO: 7 - protein, 94 aa, Artificial Sequence
  Modified Sequence
MGSSHHHHHHSSGLVPRGSHMSRIVINAYAKATQPDFKSHMFVNQHLCGSHLVEALYLVCGERGFFYTPKTRRGIVEQCCTSICSLYQLENYCG

SEQ ID NO: 8 - protein, 88 aa, Artificial Sequence
  Modified Sequence
MEKHTKDQIIEAPHMSRIVINAYAKATQPDFKSHMFVNQHLCGSHLVEALYLVCGERGFFYTPKTRRGIVEQCCTSICSLYQLENYCG

SEQ ID NO: 9 - DNA, 60 bp, Artificial Sequence
  Modified Sequence
atgagccgtattgttattaacgcgtatgcgaaagcgacccagccggattttaaaagccac

SEQ ID NO: 10 - DNA, 102 bp, Artificial Sequence
  Modifed Sequence
atggaaaaacacactaaagatcaaatcattgaagcaccgcatatgagccgtattgttattaacgcgtatgcgaaagcgacccagccggattttaaaagccac

SEQ ID NO: 11 - DNA, 288 bp, Artificial Sequence
  Modified Sequence
atgggcagcagccatcatcatcatcatcacagcagcggcctggtgccgcgcggcagccatatgagccgtattgttattaacgcgtatgcgaaagcgacccagccggattttaaaagccacatgtttgttaatcagcatctgtgcggtagccatctggtggaagcgctgtatctggtgtgtggcgaacgtggtttcttttataccccgaaaaccaaacgtggcattgtggaacagtgctgcaccagcatctgcagcctgtatcagctggaaaactattgcaattaataa

SEQ ID NO: 12 - DNA, 270 bp, Artificial Sequence
  Modified Sequence
atggaaaaacacactaaagatcaaatcattgaagcaccgcatatgagccgtattgttattaacgcgtatgcgaaagcgacccagccggattttaaaagccacatgtttgttaatcagcatctgtgcggtagccatctggtggaagcgctgtatctggtgtgtggcgaacgtggtttcttttataccccgaaaaccaaacgtggcattgtggaacagtgctgcaccagcatctgcagcctgtatcagctggaaaactattgcaattaataa

SEQ ID NO: 13 - DNA, 288 bp, Artificial Sequence
  Modified Sequence
atgggcagcagccatcatcatcatcatcacagcagcggcctggtgccgcgcggcagccatatgagccgtattgttattaacgcgtatgcgaaagcgacccagccggattttaaaagccacatgtttgttaatcagcatctgtgcggtagccatctggtggaagcgctgtatctggtgtgtggcgaacgtggtttcttttataccaaaccgaccaaacgtggcattgtggaacagtgctgcaccagcatctgcagcctgtatcagctggaaaactattgcaattaataa

SEQ ID NO: 14 - DNA, 270 bp, Artificial Sequence
  Modified Sequence
atggaaaaacacactaaagatcaaatcattgaagcaccgcatatgagccgtattgttattaacgcgtatgcgaaagcgacccagcctgattttaaaagccacatgtttgttaatcagcatctgtgcggtagccatctggtggaagcgctgtatctggtgtgtggcgaacgtggtttcttttataccaaaccgaccaaacgtggcattgtggaacagtgctgcaccagcatctgcagcctgtatcagctggaaaactattgcaattaataa

SEQ ID NO: 15 - DNA, 288 bp, Artificial Sequence
  Modified Sequence
atgggcagcagccatcatcatcatcatcacagcagcggcctggtgccgcgcggcagccatatgagccgtattgttattaacgcgtatgcgaaagcgacccagccggattttaaaagccacatgtttgttaatcagcatctgtgcggtagccatctggtggaagcgctgtatctggtgtgtggcgaacgtggtttcttttataccccgaaaacccgtcgtggcattgtggaacagtgctgcaccagcatctgcagcctgtatcagctggaaaactattgcggctaataa

SEQ ID NO: 16 - DNA, 270 bp, Artificial Sequence
  Modified Sequence
atggaaaaacacactaaagatcaaatcattgaagcaccgcatatgagccgtattgttattaacgcgtatgcgaaagcgacccagccggattttaaaagccacatgtttgttaatcagcatctgtgcggtagccatctggtggaagcgctgtatctggtgtgtggcgaacgtggtttcttttataccccgaaaacccgtcgtggcattgtggaacagtgctgcaccagcatctgcagcctgtatcagctggaaaactattgcggctaataa

(19) United States
(12) Patent Application Publication
(10) Pub. No.: US 2021/0230659 A1
(43) Pub. Date: Jul. 29, 2021

(54) Leader Sequence for Higher Expression of Recombinant Proteins

(71) Applicant: UNICHEM LABORATORIES LTD, Mumbai (IN)

(72) Inventors: Dhananjay Sathe, Thane (IN); Sudeep Kumar, Ahmedabad (IN); Sachin Prabhakar Bachate, Solapur (IN); Saikumar Kompelli, Suryapet (IN); Rahul Subhash Chougule, Tiswadi (IN)

(21) Appl. No.: 17/053,596

(22) PCT Filed: Jun. 18, 2019
(86) PCT No.: PCT/IB2019/055080

§ 371 Date: Nov. 6, 2020

(30) Foreign Application Priority Data
Jun. 18, 2018 (IN) 201821022673

(51) Int. Cl. C12P 21/02; C07K 14/62

(52) U.S. Cl. CPC C12P 21/02; C07K 14/62

(57) ABSTRACT

The present invention relates to the leader sequence for higher expression of recombinant proteins. The invention further relates to the process for preparation of insulin and insulin analogues using leader sequence. The leader peptides significantly increase the expression of pre-proinsulin. The present invention also relates to the protein sequences prepared by fusion of fragments with the leader sequences of the present invention. The invention is demonstrated by preparing and Insulin and its analogues using said leader sequences.

### FIELD OF INVENTION

The present invention relates to novel leader sequence for expression of recombinant proteins. The present invention also relates to the method of improving the expression of recombinant protein using leader sequence.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

The application of recombinant DNA technology has made a number of recombinant therapeutic proteins available for biopharmaceutical use. Both prokaryotic and eukaryotic expression systems are generally used for the recombinant protein production.

Among all expression systems, Escherichia coli (E. coli) remains the most advantageous host for producing recombinant proteins, because of its faster, inexpensive and high yielding protein production. The well-known genetics and availability of a variety of molecular tools also greatly boosted the application of E. coli in biopharmaceutical industry. Availability of a variety of promoters, leader partners and mutant strains added great advantage to E. coli to become one of the most widely used methods for recombinant protein production, both at the laboratory and industrial levels.

Along with lot of advantages, the E. coli has, however, limitations at expressing more complex proteins due to lack of sophisticated machinery to perform post translational modifications, such as glycosylation and refolding, in order to exhibit activity.

On the other hand, many mammalian proteins and other proteins cannot be expressed successfully in E. coli, which explore expression in a wide range of other organisms like Baculovirus expression system, Gram positive organisms, Pseudomonas expression systems. Higher protein production in E. coli is a major bottleneck in the process of producing recombinant proteins and many attempts have been made to overcome and resolve the issues. In some cases, researchers have explored usage of strong promoters, addition of sucrose and betaine to growth medium, use of rich medium with phosphate buffer and use of leader sequences to increase expression. Apart from lower expression, proteolytic degradation of recombinant proteins is major problem in expression host.

Additional factors to obtain high yields of protein include gene of interest, expression vector, gene dosage, transcriptional regulation, codon usage, translation regulation, host design, growth media and culture condition or fermentation conditions available for manipulating the expression conditions, specific activity or biological activity of the protein of interest, protein targeting, fusion proteins, molecular chaperons and protein degradation.

One of the best methods to increase expression and stability of expressed protein is N- or C-terminal fusions with leader sequence. Formation of strong secondary structures in transcribed mRNA reduces expression of heterologous genes. The strong secondary structure interferes with the binding of ribosomes with mRNA, thereby prevent efficient translation initiation. Leader sequence determinant at both N- and C-termini of protein can influence the recombinant protein expression and stability towards protease degradation.

Leader sequences are highly efficient tools for protein expression. Besides expression, leader sequences also have an impact on solubility and even the folding of their fusion partners. They allow the purification of virtually any protein without any requirement of any prior knowledge of its biochemical properties.

U.S. Ser. No. 10/000,544 describes a process for production of insulin or insulin analogues by expression of insulin or insulin analogues through an expression construct in a host cell. An expression construct has a leader peptide for insulin in a host cell, particularly in a bacterial cell.

U.S. Pat. No. 6,841,361 describes the use of DNA for the preparation of insulin from the fusion protein, which is obtained by the expression of the DNA through the action of thrombin and carboxypeptidase B.

JP-B-7-121226 and JP2553326 describes the method for expressing mini-proinsulin comprising a B chain and an A chain linked via two basic amino acid residues, in yeast; and then treating the mini-proinsulin with trypsin in vitro, thereby producing insulin.

However, no single leader sequence is optimal with respect to all of these parameters; each has its advantages and disadvantages. Multiple leader sequences can be added together in different combination for a particular protein to get better result with respect to expression, solubility and purification. Thus, there is a need in the art to provide leader sequences that help in efficient expression of recombinant insulin with ease and efficiency.

### OBJECT OF THE INVENTION

The main object of the present invention is to provide an efficient, novel leader sequence for expressing insulin, specifically recombinant human insulin and insulin analogues with ease and efficiency.

Another object of the present invention is to provide a fused protein comprising the novel leader sequence and proinsulin or proinsulin analogues.

A further objective of the present invention is to provide a process for preparing the fusion protein comprising the novel leader sequence and proinsulin or proinsulin analogues.

Yet another object of the present invention is to provide an easy, highly efficient and industrially scalable process to prepare insulin using the leader sequence.

Yet another object of the present invention is to provide a highly efficient process to prepare insulin or insulin analogues from pre-proinsulin comprising leader sequence.

### SUMMARY OF INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

In an aspect, the present invention relates to a leader peptide sequence selected from:

a) the peptide having amino acid sequence as set forth in SEQ ID NO: 1;
b) the peptide having amino acid sequence as set forth in SEQ ID NO: 2;
c) a peptide comprising amino acid sequence of: MSRIVINAYAKATQP;
d) a peptide comprising amino acid sequence of: MEKHTKDQIIEAPHM; or
e) a peptide having at least 80% homology to a), b), c), or d).

In another aspect, the present disclosure provides a nucleotide sequence encoding leader peptide sequence disclosed herein.

In another aspect, the present disclosure provides a nucleotide sequence selected from SEQ ID NO: 9 or SEQ ID NO: 10.

In a further aspect, the present disclosure provides a pre-proinsulin polypeptide comprising the leader peptide sequence disclosed herein which is operably linked to the precursor of insulin or insulin analogues.

In another aspect, the present disclosure provides a pre-proinsulin polypeptide of Formula 1: R₁—X₁-X₂-X₃, wherein X₁ is a ‘B’ chain of insulin or insulin analogues, X₂ is a dipeptide selected RR or KR or RK or KK, X₃ is an ‘A’ chain of insulin or insulin analogues and R1 is the leader peptide.

In another aspect, the present disclosure provides a precursor of insulin or insulin analogues which is a proinsulin of Formula 2: X₁-X₂-X₃, wherein X₁ is a ‘B’ chain of insulin or insulin analogues, X₂ is a dipeptide selected RR or KR or RK or KK and X₃ is the ‘A’ chain of insulin or insulin analogues.

In yet another aspect of the present disclosure, the leader peptide directs the expression of the insulin and insulin analogues into the prokaryotic host cell.

In another aspect of the present disclosure, the prokaryotic host cell is selected from Pseudomonas cell or Escherichia coli cell.

In an aspect, the present disclosure provides a proinsulin prepared using pre-proinsulin of Formula 1: R₁—X₁-X₂-X₃, wherein X₁ is a ‘B’ chain of insulin or insulin analogues, X₂ is a dipeptide selected RR or KR or RK or KK, X₃ is an ‘A’ chain of insulin or insulin analogues and R1 is the leader peptide.

In another aspect, the present disclosure provides a process to prepare proinsulin from pre-proinsulin, wherein the pre-proinsulin comprises the leader peptide.

In still another aspect, the present disclosure provides a process to prepare proinsulin from pre-proinsulin, wherein the pre-proinsulin is of Formula 1: R₁—X₁-X₂-X₃ and proinsulin is of formula X₁-X₂-X₃, wherein R₁ is the leader peptide, X₁ is a ‘B’ chain of insulin or insulin analogues, X₂ is a dipeptide selected RR or KR or RK or KK and X₃ is an ‘A’ chain of insulin or insulin analogues.

In an aspect, the present disclosure provides a nucleotide sequence encoding pre-proinsulin polypeptide comprising the leader peptide sequence disclosed herein which is operably linked to the precursor of insulin or insulin analogues.

In another aspect, the present disclosure provides a nucleotide sequence encoding pre-proinsulin polypeptide comprising the leader peptide sequence, wherein the nucleotide sequence is as set forth in SEQ ID NO: 11, SEQ ID NO: 12, SEQ ID NO: 13, SEQ ID NO: 14, SEQ ID NO: 15 or SEQ ID NO: 16.

In an aspect, the present disclosure provides a recombinant gene construct comprising nucleotide sequence encoding pre-proinsulin polypeptide comprising the leader peptide sequence disclosed herein or the nucleotide sequence as set forth in SEQ ID NO: 11, SEQ ID NO: 12, SEQ ID NO: 13, SEQ ID NO: 14, SEQ ID NO: 15 or SEQ ID NO: 16.

In another aspect, the present disclosure provides a recombinant gene construct wherein the gene construct is selected from pET28aULL1INS, pET28aULL2INS, pET28aULL1LSP, pET28aULL2LSP, pET28aULL1GR or pET28aULL2GR.

In an aspect, the present disclosure provides a process to prepare a recombinant gene construct comprising a nucleotide sequence encoding pre-proinsulin polypeptide comprising the leader peptide sequence disclosed herein or the nucleotide sequence as set forth in SEQ ID NO: 11, SEQ ID NO: 12, SEQ ID NO: 13, SEQ ID NO: 14, SEQ ID NO: 15 or SEQ ID NO: 16 or a gene construct selected from pET28aULL1INS, pET28aULL2INS, pET28aULL1LSP, pET28aULL2LSP, pET28aULL1GR or pET28aULL2GR.

In a further aspect, the present disclosure provides an expression vector comprising a gene construct comprising a nucleotide sequence encoding pre-proinsulin polypeptide comprising the leader peptide sequence disclosed herein or the nucleotide sequence as set forth in SEQ ID NO: 11, SEQ ID NO: 12, SEQ ID NO: 13, SEQ ID NO: 14, SEQ ID NO: 15 or SEQ ID NO: 16 or a gene construct selected from pET28aULL1INS, pET28aULL2INS, pET28aULL1LSP, pET28aULL2LSP, pET28aULL1GR or pET28aULL2GR.

In another aspect, the present disclosure provides an expression vector wherein the vector comprises the recombinant gene construct pET28aULL1INS or pET28aULL2INS for production of insulin, pET28aULL1LSP or pET28aULL2LSP for production of insulin Lispro and pET28aULL1GR or pET28aULL2GR for production of insulin glargine.

In still another aspect, the present disclosure provides a prokaryotic host cell comprising an expression vector disclosed herein.

In another aspect, the present disclosure provides a prokaryotic host cell comprising an expression vector selected from Pseudomonas cell or Escherichia coli cell.

In an aspect, the present disclosure provides a method of expressing an insulin and insulin analogue via expression of proinsulin as disclosed herein.

In another aspect, the present invention provides a method of expressing an insulin and insulin analogue via expression of proinsulin wherein the method comprises fermentation of the prokaryotic host cell in a suitable production medium.

In still another aspect, the present invention provides a method of expressing an insulin and insulin analogue via expression of proinsulin, wherein the production medium comprises 1% yeast extract, 1% Dextrose, 0.3% KH₂PO₄, 1.25% K₂HPO₄, 0.5% (NH₄)₂SO₄, 0.05% NaCl, 0.1% MgSO₄.7H₂O, 0.1% of trace metal solution (FeSO₄, ZnSO₄, CoCl₂, NaMoO₄, CaCl₂, MnCl₂, CuSO₄ or H₃BO₃ in Hydrochloric acid) and Kanamycin (20 μg/ml) per 100 ml.

In a further aspect, the present disclosure provides a process to produce insulin and insulin analogues, wherein the process comprises use of leader peptide disclosed herein.

In another aspect, the present disclosure provides a process to produce insulin and insulin analogues, wherein the process comprises use of pre-proinsulin polypeptide comprising the leader peptide sequence disclosed herein which is operably linked to the precursor of insulin or insulin analogues or the polypeptide.

In yet another aspect, the present disclosure provides a process to produce insulin and insulin analogues, wherein the process comprises use of proinsulin disclosed herein.

In an aspect, the present disclosure provides insulin or insulin analogues prepared by the process comprising leader peptide disclosed herein.

In another aspect, the present disclosure provides insulin or insulin analogues prepared by the process comprising pre-proinsulin polypeptide comprising the leader peptide sequence disclosed herein which is operably linked to the precursor of insulin or insulin analogues.

In another aspect, the present disclosure provides insulin or insulin analogues prepared by the process comprising proinsulin as disclosed herein.

These and other features, aspects, and advantages of the present invention will be better understood with reference to the following description and appended claims. Other aspects of the invention will be set forth in the description which follows, and in part will be apparent from the description, or may be learnt by the practice of the invention.

### BRIEF DESCRIPTION OF THE DRAWINGS

The following drawings form part of the present specification and are included to further illustrate aspects of the present disclosure. The disclosure may be better understood by reference to the drawings in combination with the detailed description of the specific embodiments presented herein.

FIG. 1: is an expression analysis of pre-proinsulin with construct pET28aULL1INS and pET28aULL2INS in E. coli BL21 DE3.

FIG. 2 is an expression analysis of pre-proinsulin-Lispro with construct pET28aULL1LSP and pET28aULL2LSP in E. coli BL21 DE3.

FIG. 3 is an expression analysis of pre-proinsulin Glargine with construct pET28aULL1GLR and pET28aULL2GLR in E. coli BL21 DE3.

FIG. 4 is an annotated diagram of pET28a Vector Map with ULL1INS.

FIG. 5 is an annotated diagram of pET28a Vector Map with ULL2INS.

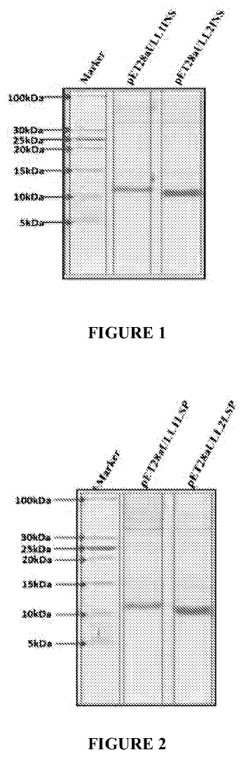

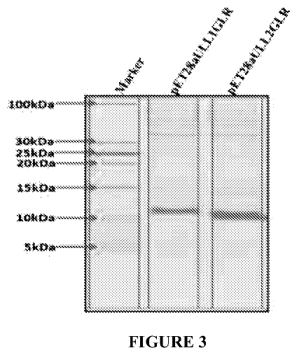

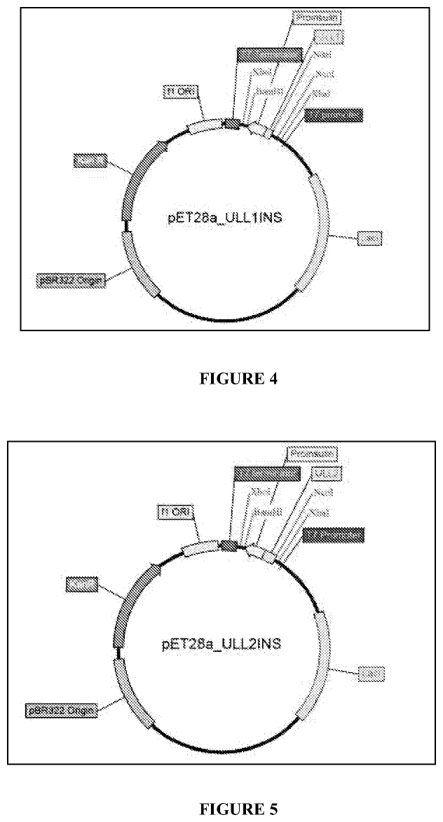

### BRIEF DESCRIPTION OF ACCOMPANYING SEQUENCES

SEQ ID NO: 1 is an amino acid sequence of ULL1, which is a leader sequence (R₁)
SEQ ID NO: 2 is an amino acid sequence of ULL2, which is a leader sequence (R₁)
SEQ ID NO: 3 is an amino acid sequence of SEQ ID NO: 1 fused to proinsulin sequence of insulin.
SEQ ID NO: 4 is an amino acid sequence of SEQ ID NO: 2 fused to proinsulin sequence of insulin.
SEQ ID NO: 5 is an amino acid sequence of SEQ ID NO: 1 fused to proinsulin sequence of insulin Lispro.
SEQ ID NO: 6 is an amino acid sequence of SEQ ID NO: 2 fused to proinsulin sequence of insulin Lispro.
SEQ ID NO: 7 is an amino acid sequence of SEQ ID NO: 1 fused to proinsulin sequence of insulin Glargine.
SEQ ID NO: 8 is an amino acid sequence of SEQ ID NO: 2 fused to proinsulin sequence of insulin Glargine.
SEQ ID NO: 9 is a nucleotide sequence encoding SEQ ID NO: 1.
SEQ ID NO: 10 is a nucleotide sequence encoding SEQ ID NO: 2.
SEQ ID NO: 11 is a nucleotide sequence encoding SEQ ID NO: 3.
SEQ ID NO: 12 is a nucleotide sequence encoding SEQ ID NO: 4.
SEQ ID NO: 13 is a nucleotide sequence encoding SEQ ID NO: 5.
SEQ ID NO: 14 is a nucleotide sequence encoding SEQ ID NO: 6.
SEQ ID NO: 15 is a nucleotide sequence encoding SEQ ID NO: 7.
SEQ ID NO: 16 is a nucleotide sequence encoding SEQ ID NO: 8.

### DETAILED DESCRIPTION OF THE INVENTION

The following is a detailed description of embodiments of the disclosure. Those skilled in the art will be aware that the present disclosure is subject to variations and modifications other than those specifically described. It is to be understood that the present disclosure includes all such variations and modifications. The detailed disclosure offered is not intended to limit the anticipated variations of embodiments; on the contrary, the intention is to cover all modifications, equivalents, and alternatives falling within the spirit and scope of the present disclosure as defined by the appended claims. The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

Where a definition or use of a term in an incorporated reference is inconsistent or contrary to the definition of that term provided herein, the definition of that term provided herein applies and the definition of that term in the reference does not apply.

Various terms as used herein are shown below. To the extent a term used in a claim is not defined below, it should be given the broadest definition persons in the pertinent art have given that term as reflected in printed publications and issued patents at the time of filing.

The term ‘Peptide’ as used herein refers to a molecule comprising an amino acid sequence connected by peptide bonds regardless of length, post-translation modification, or function.

The term “Dipeptide” as used herein refers to a molecule comprising an amino acid sequence of two (2) amino acids connected by peptide bonds.

The term “Polypeptide” as used herein refers to naturally occurring or recombinant, produced or modified chemically or by other means, which may assume the three dimensional structure of proteins that may be post-translationally processed, essentially the same way as native proteins.

The terms ‘peptides’, ‘polypeptide’ and ‘protein’ are used interchangeably herein.

The term “Insulin” as used herein refers to a hormone which is 51 amino acid residue polypeptide (5808 Daltons), which plays an important role in many key cellular processes. It is involved in the stimulation of cell growth and differentiation. It also exerts its regulatory function (e.g. uptake of glucose into cells) through a signalling pathway initiated by binding of hormone in its monomeric form to its dimeric, tyrosine-kinase type membrane receptor. The mature form of human insulin consists of 51 amino acids arranged into an A-chain (GlyA1-AsnA21) and a B chain (PheB1-ThrB30) of total molecular mass of 5808 Da. The molecule is stabilised by two inter (A20-B19, A7-B7) and one intra chain disulphide bonds (A6-A11). Insulins of the present invention include natural, provided by synthetic, or genetically engineered (e.g., recombinant) sources, in various embodiments of the present invention, insulin can be a human insulin.

The term “insulin analogues” as used herein refers to altered form of insulin which is either a more rapid acting or more uniformly acting form of the insulin. Non-limiting examples of such analogues are Insulin Lispro, Insulin Degludec, Insulin Aspart and Insulin Glargine. Insulin Analogue “Lispro” is identical in primary structure to human insulin, differs from human insulin by switching the lysine at position B28 and the proline at position B29. It is a short-acting insulin monomeric analogue. Insulin Analogue “Glargine” differs from human insulin by a substitution of asparagine for glycine at A21, and the addition of two arginine residues to the C-terminus of the B-chain. Insulin glargine solution is formulated and injected at pH 4.0. These modifications increase the isoelectric point to a more neutral pH, reducing the solubility under physiologic conditions and causing glargine to precipitate at the injection site, thus slowing absorption. Glargine is an extended-action analogue that lasts 20-24 hour.

The term ‘Pre-proinsulin’ as used herein refers to a single chain polypeptide molecule comprising a leader peptide (R₁), a B chain (X₁) of Insulin, a C-peptide or dipeptide (X₂) and A chain (X₃) of Insulin, linked in the order represented by the formula “R₁—X₁-X₂-X₃”.

The terms ‘pre-proinsulin’ or ‘preproinsulin’ are used interchangeably herein.

The term ‘Proinsulin’ as used herein refers to a single chain polypeptide molecule generated after cleavage of leader sequence from pre-proinsulin and is represented by the formula X₁—X₂-X₃, which includes the dipeptide or “C-peptide”(X₂) linking the B chain(X₁) and A chain(X₃) of insulin.

The term “nucleic acid sequence” or polynucleotide sequence as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The nucleic acid sequences encoding insulin that may be used in accordance with the methods provided herein may be any nucleic acid sequence encoding an insulin polypeptide or its precursors including proinsulin and pre-proinsulin.

The term “operably linked” as used herein refers to a configuration in which a control sequence, which herein is the leader sequence R₁ is placed at an appropriate position relative to the coding sequence of the polynucleotide sequence such that the control sequence directs the expression of the coding sequence to the polypeptide.

The term “coding sequence” as used herein refers to a polynucleotide sequence that is transcribed into mRNA which is translated into a polypeptide when placed under the control of the appropriate control sequences, which herein is the leader sequence R₁. The boundaries of the coding sequence are generally determined by the start codon located at the beginning of the open reading frame of the 5′ end of the mRNA and a stop codon located at the 3′ end of the open reading frame of the mRNA. A coding sequence may include, but is not limited to, genomic DNA, cDNA, semi-synthetic, synthetic, and recombinant nucleotide. The coding sequence for example is the nucleotide sequence encoding proinsulin of formula X₁-X₂-X₃

The term ‘pET28aULL1INS’ as used herein refers to the plasmid used to encode pre-proinsulin using vector pET28a, nucleotide sequence of SEQ ID 9 and the nucleotide sequence encoding X₁-X₂-X₃ corresponding recombinant human Insulin as defined herein before.

The term ‘pET28aULL1LSP’ as used herein refers to the plasmid used to encode pre-proinsulin using vector pET28a, nucleotide sequence of SEQ ID 9 and the nucleotide sequence encoding X₁-X₂-X₃ corresponding Insulin Lispro as defined herein before.

The term ‘pET28aULL1GR’ as used herein refers to the plasmid used to encode pre-proinsulin using vector pET28a, nucleotide sequence of SEQ ID 9 and the nucleotide sequence encoding X1-X2-X3 corresponding Insulin Glargine as defined herein before.

The term ‘pET28aULL2INS’ as used herein refers to the plasmid used to encode pre-proinsulin using vector pET28a, nucleotide sequence of SEQ ID 10 and the nucleotide sequence encoding X1-X2-X3 corresponding recombinant human Insulin as defined herein before.

The term ‘pET28aULL2LSP’ as used herein refers to the plasmid used to encode pre-proinsulin using vector pET28a, nucleotide sequence of SEQ ID 10 and the nucleotide sequence encoding X1-X2-X3 corresponding Insulin Lispro as defined herein before. The term ‘pET28aULL2GR’ as used herein refers to the plasmid used to encode pre-proinsulin using vector pET28a, nucleotide sequence of SEQ ID 10 and the nucleotide sequence encoding X1-X2-X3 corresponding Insulin Glargine as defined herein before.

The terms “leader sequence” or “Tag” as used herein refers to peptide sequence located at the amino terminal of the precursor form of a protein, which maximizes the production of protein.

The present invention provides a sequence having at least 80% homology to amino acid sequence as set forth in SEQ ID NO: 1 and SEQ ID NO: 2. The amino acid sequences as set forth in SEQ ID NO: 1 and SEQ ID NO: 2 are also referred to as ULL1 and ULL2, respectively.

The present invention provides a process for producing insulin, more specifically, human insulin and insulin analogues. The invention also relates to a peptide used in the present process for higher expression.

In an embodiment of the present invention, there is provided pre-proinsulin sequences and processes for the preparation of insulin and insulin analogues from pre-proinsulin sequences via proinsulin, wherein the said pre-proinsulin of Formula 1 and proinsulin of Formula 2 are as follows:

R1-X1-X2-X3 and Formula 2: X1-X2-X3, Formula 1:

wherein R1 is peptide having amino acid sequence as set forth in SEQ ID NO: 1 or peptide having amino acid sequence as set forth in SEQ ID NO: 2.

X1 is ‘B’ chain of insulin and insulin analogues,

X2 is dipeptide comprising RR or KR or RK or KK, and

X3 is ‘A’ chain of insulin and insulin analogues.

In an embodiment of the present invention, the peptide has amino acid sequence as set forth in SEQ ID NO: 1 and an amino acid sequence as set forth in SEQ ID NO: 2. The peptides having amino acid sequences as set forth in SEQ ID NO: 1 and SEQ ID NO: 2 are also called as a leader sequence or a Tag. The novel sequences of SEQ ID NO: 1 and SEQ ID NO: 2 disclosed in the present invention enhance expression of proteins such as low molecular weight proteins in bacterial host cells and thus leads to higher yields of proteins of interest. As is well known, the expression of low molecular weight proteins in bacterial host cell is difficult due the unstable messenger RNA and rapid degradation of these proteins. Inefficient translation of the underlying coding sequences also leads to lower expression of low molecular weight proteins. The novel sequences disclosed in the present invention attempt to overcome these drawbacks prevalent in the art.

Another embodiment of the invention provides a peptide having at least 80% homology to the sequence of amino acids from 1 to 15 as set forth in SEQ ID NO: 1 or SEQ ID NO: 2.

In an embodiment of the present invention, the leader sequences having amino acid sequences as set forth in SEQ ID NO: 1 and SEQ ID NO: 2 were designed by considering the important factors for the higher expression of recombinant protein. The factors which affect the recombinant protein expression in bacterial host cell are: size of the protein, GC content of the coding DNA sequence, mRNA secondary structure, translation initiation rate and codon usage of bacterial host cell. The factors considered were GC content of the coding DNA sequence, mRNA secondary structure, translation initiation rate and codon usage of bacterial host cell.

In an embodiment of the present invention, the host cells were preferably E. coli, and more preferably E. coli Gold BL 21 DE3.

In an embodiment of the present invention, the gene encoding the proinsulin having nucleotide sequence as set forth in SEQ ID NO: 9 encoding the peptide of SEQ ID NO: 1 was designed, codon optimized, chemically synthesized and cloned in pUC57 by Genscript® to prepare pUC57ULL1INS. Restriction digestion of pUC57ULL1INS plasmid and pET28a vector was done using NdeI and BamH1 restriction enzymes. Gene fragment, ULL1INS was purified by gel elution kit (Qiagen®) and was ligated to pET28a vector to prepare pET28aULL1INS. Further it was transformed into propagation host, E. coli TOP10 cells to propagate pET28aULL1INS, ligated plasmid. Such plasmid was isolated and transformed into E. coli Gold BL 21 DE3 cells to check the expression of protein.

In another embodiment of the present invention, the gene encoding the proinsulin comprising nucleotide sequence as set forth in SEQ ID NO: 10 encoding the peptide of SEQ ID NO: 2 was designed, codon optimized and chemically synthesized and cloned in pUC57 by Genscript® to prepare pUC57ULL2INS. Restriction digestion of pUC57ULL2INS plasmid and pET28a vector was done using NcoI and BamH1 restriction enzymes. Gene fragment, ULL2INS was purified by gel elution kit (Qiagen®) and was ligated to pET28a vector to prepare pET28aULL2INS. Further it was transformed into propagation host, E. coli TOP10 cells to propagate pET28aULL2INS, ligated plasmid. Such plasmid was isolated and transformed into E. coli Gold BL 21 DE3 cells to check the expression of protein.

In a further embodiment of the present invention, there is provided gene constructs for the preparation of insulin analogues such as insulin Glargine and insulin Lispro.

The insulin fragment used in the present invention has 159 bp in length and corresponds to the nucleotide sequence of the insulin protein with the small C-chain (2 amino acids) thereof.

In an aspect of the present invention, a process for preparing insulin from pre-proinsulin sequence is provided. The process comprises the following steps of fermentation, cell lysis, inclusion bodies preparation, solubilization of inclusion bodies, cleavage of leader peptide to obtain proinsulin, anion exchange chromatography, refolding, hydrophobic interaction chromatography, enzymatic cleavage by trypsin, anion/cation exchange chromatography, enzymatic cleavage by carboxypeptidase and reverse phase chromatography.

In an embodiment of the present invention, the process for preparing insulin from pre-proinsulin comprises fermentation step which comprises growing the E. coli cells transformed with pET28aULL1INS or pET28aULL2INS in a production medium, inducing with Isopropyl β-D-1-thiogalactopyranoside (IPTG) and harvesting the cell mass obtained at the end of the fermentation process.

In an embodiment of the present invention, the process for preparing insulin from pre-proinsulin comprises cell lysis step. The cells containing inclusion bodies of pre-proinsulin were re-suspended in Tris-NaCl buffer and lysed by high pressure with Mini-DeBEE homogenizer.

In an embodiment of the present invention, the process for preparing insulin from pre-proinsulin comprises the step of inclusion bodies preparation. The inclusion bodies enriched with pre-proinsulin were washed with Tris-NaCl buffer containing reducing agent such as β-mercaptoethanol.

In an embodiment of the present invention, the process for preparing insulin from pre-proinsulin comprises the step of solubilization of inclusion bodies. The inclusion bodies were dissolved in 6M guanidine hydrochloride in basic buffer. The dissolved inclusion bodies suspension was subjected to sulfitolysis by adding sodium sulfite and sodium tetrathionate.

In an embodiment of the present invention, the process for preparing insulin from pre-proinsulin comprises the step of cleaving the leader peptide to obtain proinsulin. The pH of the solubilized inclusion bodies suspension was adjusted to 1-2. Cyanogen bromide was added to the solution and incubated at 8° C. overnight. The protein was then precipitated by adding excess purified water and then the pellet obtained after centrifugation is washed with glycine buffer and dissolved in 8M urea.

In an embodiment of the present invention, the process for preparing insulin from pre-proinsulin comprises the step of anion exchange chromatography. The protein dissolved in 8M urea was subjected to anion exchange chromatography. The protein was loaded on anion exchange resin and eluted with 8M urea buffer containing sodium chloride. The proinsulin was obtained in concentrated form.

In an embodiment of the present invention, the process for preparing insulin from pre-proinsulin comprises the step of refolding. The proinsulin obtained in the concentrated form was then subjected to refolding by dilution in glycine buffer. The pH of the solution was maintained at 9.5 and protein concentration was in the range of 0.5 to 1 mg/ml. The refolding reaction was allowed to proceed at 25° C. for 2-3 hours. The reaction was stopped by addition of acetic acid so as to bring the pH to ˜4.0.

In an embodiment of the present invention, the process for preparing insulin from pre-proinsulin comprises the step of hydrophobic interaction chromatography (HIC). The refolded solution was subjected to hydrophobic interaction chromatography. The conductivity of the solution was increased by addition of sodium chloride and then protein was loaded onto hydrophobic interaction resin. The proinsulin was eluted with the increasing gradient of sodium chloride in glycine buffer.

In an embodiment of the present invention, the process for preparing insulin from pre-proinsulin comprises the step of enzymatic cleavage by trypsin. Protein eluted from HIC was digested with 1:5000 ratio of protein to trypsin. Preferably, the trypsin is in a powder form or immobilized form. When immobilized trypsin is used, the reaction is stopped by separating the beads containing trypsin by filtration. When powder form of trypsin is used, the reaction is quenched by addition of acetic acid.

In an embodiment of the present invention, the process for preparing insulin from pre-proinsulin comprises the step of Anion/Cation exchange chromatography. Based on the form of trypsin used (powder or immobilized) for cleaving, the protein can be subjected to either cation or anion exchange chromatography. Preferably, the protein is eluted by increasing gradient of sodium chloride.

In an embodiment of the present invention, the process for preparing insulin from pre-proinsulin comprises the step of enzymatic cleavage by carboxypeptidase. The protein eluted from the exchange chromatography is digested with carboxypeptidase to remove C-terminal arginine from B-chain.

In an embodiment of the present invention, the process for preparing insulin from pre-proinsulin comprises the step of Reverse phase chromatography. The active insulin is purified from the digested sample by reverse phase chromatography. The protein is loaded to achieve final binding in the range of 10-15 mg/ml of resin. Preferably, the insulin is eluted using increasing gradient of acetonitrile.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The examples given below are strictly for illustration purposes only and do not limit the invention in any manner. Various modifications of the disclosed embodiments, as well as alternate embodiments of the said invention, will become apparent to the person skilled in the art. It is therefore contemplated that such modifications can be made without departing from the true spirit or scope of the present invention as exemplified here.

### Example 1: Construction of Plasmid pET28aULL1INS

Gene encoding proinsulin along with nucleotide sequence of SEQ ID NO: 9 coding for peptide ULL1INS, was designed, codon optimized and chemically synthesized and cloned in pUC57 by Genescript® to prepare pUC57ULL1INS. Gene fragment was cloned into pET28a vector. Restriction digestion of pUC57ULL1INS plasmid was done by setting up reaction mix having plasmid 10 μl, NdeI 1 μl, BamHI 1 μl, 10×NEB buffer 2 μl and sterile water 6 μl. pET28a vector subjected to restriction digestion by enzymes NdeI and BamHI to produce sticky ends. Reaction mix contained 10 μl pET28a vector, 1 μl NdeI, 1 μl BamHI, 2 μl 10×NEB buffer and 6 μl sterile water. Both reactions were incubated at 37° C. for 2 hours. Gene fragment was purified by gel elution kit (Qiagen®) and was ligated to pET28a vector. Further it was transformed into propagation host, E. coli TOP10 cells to propagate ligated plasmids. Such plasmid was isolated and transformed into E. coli Gold BL 21 DE3 cells to check the expression of protein.

### Example 2: Construction of Plasmid pET28aULL2INS

The gene encoding the proinsulin along with nucleotide sequence of SEQ ID NO: 10 coding for peptide ULL2INS was designed, codon optimized and chemically synthesized and cloned in pUC57 by Genscript® to prepare pUC57ULL2INS. Gene fragment was cloned into pET28a vector. Restriction digestion of pUC57ULL2INS plasmid was done by setting up reaction mix having 10 μl plasmid, 10 NcoI, 10 BamHI, 2 μl 10×NEB buffer and 6 μl sterile water pET28a vector subjected to restriction digestion by enzymes NcoI and BamHI to produce sticky ends. Reaction mix contained pET28a vector 10 μl, NcoI 10, BamHI 10, 10×NEB buffer 2 μl and sterile water 6 μl. Both reactions were incubated at 37° C. for 2 hours. Gene fragment was purified by gel elution kit (Qiagen®) and was ligated to pET28a vector Further it was transformed into propagation host, E. coli TOP10 cells to propagate ligated plasmids. Such plasmid was isolated and transformed into E. coli Gold BL 21 DE3 cells to check the expression of protein.

### Example 3: Construction of Plasmid pET28aULL1LSP

To obtain construct pET28aULL1LSP, PCR based Site Directed Mutagenesis was done in plasmid pET28aULL1INS. Site directed mutagenesis would bring change at B28 and B29 position of B chain from PK to KP. Following pair of mutagenesis primers was used

Forward:
5′ GTG GTT TCT TTT ATA CCA AAC CGA CCA AAC GTG
GCA TTG T 3′
Reverse:
5′ ACA ATG CCA CGT TTG GTC GGT TTG GTA TAA AAG
AAA CCA C 3′

PCR reaction mix consisted of 300 μM dNTP mix, 1×PFu buffer, 10 pm each primer, 1 μl template plasmid and 41 μl sterile water. PCR condition used were: 94° C.-8 mins, 94° C.-40 sec, 55° C.-40 sec, 68° C.-3 mins (20 cycles) and 68° C. for 10 mins. Site directed mutagenesis product was subjected to DpnI digestion and then transformed into propagation host, E. coli TOP10 cells for propagation. Plasmid was isolated using Fermentas® miniprep kit and then transformed into E. coli Gold BL 21 DE3 cells for expression of protein.

### Example 4: Construction of Plasmid pET28aULL2LSP

To obtain construct pET28aULL2LSP, PCR based Site Directed Mutagenesis was done in plasmid pET28aULL2INS. Site directed mutagenesis would bring change at B28 and B29 position of B chain from PK to KP. Following pair of mutagenesis primers was used

Forward:
5′ GTG GTT TCT TTT ATA CCA AAC CGA CCA AAC GTG
GCA TTG T 3′
Reverse:
5′ ACA ATG CCA CGT TTG GTC GGT TTG GTA TAA AAG
AAA CCA C 3′

PCR reaction mix consisted of 300 μM dNTP mix, 1×PFu buffer, 10 pm each primer, 1 μl template plasmid and 410 sterile water. PCR programme was kept as follows: 94° C. for 8 mins, 94° C. for 40 sec, 55° C. for 40 sec, 68° C. for 3 mins (20 cycles) and final extension at 68° C. at 10 mins. Site directed mutagenesis product was subjected to DpnI digestion and then transformed into propagation host, E. coli TOP10 cells for propagation. Plasmid was isolated using Fermentas® miniprep kit and then transformed into E. coli Gold BL 21 DE3 cells for expression of protein.

### Example 5: Construction of Plasmid pET28aULL1GR

To obtain the construct pET28aULL1GR a site directed mutagenesis in plasmid pET28aULL1INS was done. Site directed mutagenesis primers would introduce additional Arg (R) at the end of B chain and replace Aspargine (N) with Glycine (G) in A chain. This would convert Insulin sequence into Glargine sequence. This was done in two step site directed mutagenesis PCR. In first SDM PCR following primers were used

Forward:
5′ AAACCGACCAAACGTCGTGGCATTGTGGAACA 3′
Reverse:
5′ TGTTCCACAATGCCACGACGTTTGGTCGGTTT 3′

PCR reaction mix consisted of 300 μM dNTP mix, 1×PFu buffer, 10 pm each primer, 1 μl template plasmid and 410 sterile water. Thermal cycler conditions used for amplification were: 94° C. for 8 mins, 94° C. for 40 sec, 55° C. for 40 sec, 68° C. for 3 mins (20 cycles) and 68° C. for 10 mins. Site directed mutagenesis product was subjected to DpnI digestion and then transformed into propagation host, E. coli TOP10 cells for propagation. Plasmid was isolated using from these colonies using Fermentas® minprep kit. This plasmid was used as template for second SDM PCR

Further following pair of mutagenesis primers was used for second step SDM PCR.

Forward:
5′ CTGGAAAACTATTGCGGCTAATAAGGATCCGAA 3′
Reverse:
5′ TTCGGATCCTTATTAGCCGCAATAGTTTTCCAG 3′

PCR reaction mix consisted of 300 μM dNTP mix, 1×PFu buffer, 10 pm each primer, 1 μl template plasmid and 410 sterile water. PCR program was kept as follows: 94° C. for 8 mins, 94° C. for 40 sec, 55° C. for 40 sec, 68° C. for 3 mins (20 cycles) and 68° C. for 10 mins. Site directed mutagenesis product was subjected to DpnI digestion and then transformed into propagation host, E. coli TOP10 cells for propagation. Plasmid was isolated using Fermentas® miniprep kit and then transformed into E. coli Gold BL 21 DE3 cells for expression of protein.

### Example 6: Construction of Plasmid pET28aULL2GR

To obtain the construct pET28aULL2GR a site directed mutagenesis in plasmid pET28aULL2INS was done. Site directed mutagenesis primer would introduce additional Arg (R) at the end of B chain and replace Asparagine (N) with Glycine (G) in A chain. This would convert Insulin sequence into Glargine sequence. This was done in two step site directed mutagenesis PCR. In first SDM PCR following primers were used

Forward:
5′ AAACCGACCAAACGTCGTGGCATTGTGGAACA 3′
Reverse:
5′ TGTTCCACAATGCCACGACGTTTGGTCGGTTT 3′

PCR reaction mix consisted of 300 μM dNTP mix, 1×PFu buffer, 10 pm each primer, 1 μl template plasmid and 410 sterile water. PCR program used for amplification was: 94° C. for 8 mins, 94° C. for 40 sec, 55° C. for 40 sec, 68° C. for 3 mins (20 cycles) and final extension at 68° C. for 10 mins. Site directed mutagenesis product was subjected to DpnI digestion and then transformed into propagation host, E. coli. TOP10 cells for propagation. Plasmid was isolated using from these colonies using Fermentas® minprep kit. This plasmid was used as template for second SDM PCR.

Further following pair of mutagenesis primers was used for second step SDM PCR.

Forward:
5′ CTGGAAAACTATTGCGGCTAATAAGGATCCGAA 3′
Reverse:
5′ TTCGGATCCTTATTAGCCGCAATAGTTTTCCAG 3′

PCR reaction mix consisted of 300 μM dNTP mix, 1×PFu buffer, 10 pm each primer, 1 μl template plasmid and 410 sterile water. PCR program used for amplification was: 94° C. for 8 mins, 94° C. for 40 sec, 55° C. for 40 sec, 68° C. for 3 mins (20 cycles) and 68° C. for 10 mins. Site directed mutagenesis product was subjected to DpnI digestion and then transformed into propagation host, E. coli. TOP10 cells for propagation. Plasmid was isolated using Fermentasminiprep kit and then transformed into E. coli Gold BL 21 DE3 cells for expression of protein.

Construct sequencing: All the constructs prepared in this work were confirmed by sequencing.

### Example 7: Expression Analysis of Insulin Using Construct pET28aULL1INS

The E. coli cells containing vector pET28aULL1INS was grown in 50 ml of Hiveg Luria broth containing 20 μg/ml kanamycin at 37° C., 160 rpm for overnight. The 2% culture was then transferred to 150 ml of production medium containing 1% yeast extract, 1% Dextrose, 0.3% KH₂PO₄, 1.25% K₂HPO₄, 0.5% (NH₄)₂SO₄, 0.05% NaCl, 0.1% MgSO₄.7H₂O and 0.1% of trace metal solution (FeSO₄, ZnSO₄, CoCl₂, NaMoO₄, CaCl₂, MnCl₂, CuSO₄ or H₃BO₃ in Hydrochloric acid). Kanamycin was added to a final concentration of 20 μg/ml. The culture was incubated at 37° C., 140 rpm. The culture was induced with 1 mM IPTG when cell density reached to 1-1.2 (OD600 nm). The culture was further incubated for 4 hours. The expression of pre-proinsulin was analyzed by SDS-PAGE analysis. The expression was pre-proinsulin was ˜25% of total cellular protein.

### Example 8: Expression Analysis of Insulin Using Construct pET28aULL2INS

The E. coli cells containing vector pET28aULL2INS was grown in 50 ml of Hiveg Luria broth containing 20 μg/ml kanamycin at 37° C., 160 rpm for overnight. The 2% culture was then transferred to 150 ml of production medium containing 1% yeast extract, 1% Dextrose, 0.3% KH₂PO₄, 1.25% K₂HPO₄, 0.5% (NH₄)₂SO₄, 0.05% NaCl, 0.1% MgSO₄.7H₂O and 0.1% of trace metal solution (FeSO₄, ZnSO₄, CoCl₂, NaMoO₄, CaCl₂, MnCl₂, CuSO₄ or H₃BO₃ in Hydrochloric acid). Kanamycin was added to a final concentration of 20 μg/ml. The culture was incubated at 37° C., 140 rpm. The culture was induced with 1 mM IPTG when cell density reached to 1-1.2 (OD600 nm). The culture was further incubated for 4 hours. The expression of pre-proinsulin was analyzed by SDS-PAGE analysis. The expression was pre-proinsulin was ˜40% of total cellular protein.

### Example 9: Preparation of Human Insulin Using the Construct pET28aULL1INS

Fermentation process—E. coli cells transformed with pET28aULL1INS were grown in production medium, induced with IPTG and cell mass is obtained at the end of fermentation process.

Cell lysis—The cells containing inclusion bodies of pre-proinsulin were re-suspended in Tris-NaCl buffer and lysed by high pressure with Mini-DeBEE homogenizer.

Inclusion bodies preparation—Inclusion bodies enriched with pre-proinsulin were washed with Tris-NaCl buffer containing reducing agent such as β-mercaptoethanol.

Solubilization of inclusion bodies-Inclusion bodies were dissolved in 6M guanidine hydrochloride in basic buffer. The dissolved inclusion bodies suspension was subjected to sulfitolysis by adding sodium sulfite and sodium tetrathionate.

Cleavage of leader peptide to obtain proinsulin—The pH of the solubilized inclusion bodies suspension was adjusted to 1-2. Cyanogen bromide was added to the solution and incubated at 8° C. overnight. The protein was then precipitated by adding excess purified water and then pellet obtained after centrifugation is washed with glycine buffer and dissolved in 8M urea.

Anion exchange chromatography—The protein dissolved in 8M urea was subjected to anion exchange chromatography. The protein was loaded on anion exchange resin and eluted with 8M urea buffer containing sodium chloride. The proinsulin was obtained in concentrated form.

Refolding—The proinsulin was then subjected to refolding by dilution in glycine buffer. The pH of the solution was maintained at 9.5 and protein concentration was in the range of 0.5 to 1 mg/ml. The refolding reaction was allowed at 25° C. for 2-3 hours. The reaction was stopped by addition of acetic acid so as to bring the pH to ˜4.0.

Hydrophobic interaction chromatography (HIC)—The refolded solution was subjected to hydrophobic interaction chromatography. The conductivity of the solution was increased by addition of sodium chloride and then protein was loaded onto hydrophobic interaction resin. The proinsulin was eluted with the increasing gradient of sodium chloride in glycin buffer.

Enzymatic cleavage by trypsin—The protein eluted from HIC was digested with 1:8000 ratio of protein to trypsin at 4° C. The reaction was monitored by HPLC and was at the completion reaction was stopped by separating the immobilized trypsin with filtration.

Anion exchange chromatography—The digested protein was further purified by anion exchange chromatography. The protein was loaded onto anion exchange chromatography and eluted with buffer containing sodium chloride. The Insulin was eluted by using increasing gradient of sodium chloride.

Enzymatic cleavage by carboxypeptidase—The protein from above step is then digested with carboxypeptidase to remove C-terminal arginine from B-chain.

Reverse phase chromatography—The active insulin is purified from digested sample by reverse phase chromatography. The protein is loaded to achieve final binding in the range of 10-15 mg/ml of resin. The insulin is eluted using increasing gradient of acetonitrile.

### Example 10: Preparation of Human Insulin Using the Construct pET28aULL2GLR

This example demonstrates the utility of the invention to produce the higher quantity of human insulin from the gene construct pET28aULL2GLR. The process followed for preparation of human insulin glargine using construct pET28aULL2INS is as described below.

Fermentation process—E. coli cells transformed with pET28aULL1INS were grown in production medium, induced with IPTG and cell mass is obtained at the end of fermentation process.

Cell lysis—The cells containing inclusion bodies of pre-proinsulin were re-suspended in Tris-NaCl buffer and lysed by high pressure with Mini-DeBEE homogenizer.

Inclusion bodies preparation—Inclusion bodies enriched with pre-proinsulin were washed with Tris-NaCl buffer containing reducing agent such as β-mercaptoethanol.

Solubilization of inclusion bodies-Inclusion bodies were dissolved in 6M guanidine hydrochloride in basic buffer. The dissolved inclusion bodies suspension was subjected to sulfitolysis by adding sodium sulfite and sodium tetrathionate.

Cleavage of leader peptide to obtain proinsulin—The pH of the solubilized inclusion bodies suspension was adjusted to 1-2. Cyanogen bromide was added to the solution and incubated at 8° C. overnight. The protein was then precipitated by adding excess purified water and then pellet obtained after centrifugation is washed with glycine buffer and dissolved in 8M urea.

Anion exchange chromatography—The protein dissolved in 8M urea was subjected to anion exchange chromatography. The protein was loaded on anion exchange resin and eluted with 8M urea buffer containing sodium chloride. The proinsulin was obtained in concentrated form.

Refolding—The proinsulin was then subjected to refolding by dilution in glycine buffer. The pH of the solution was maintained at 9.5 and protein concentration was in the range of 0.5 to 1 mg/ml. The refolding reaction was allowed at 25° C. for 2-3 hours. The reaction was stopped by addition of acetic acid so as to bring the pH to ˜4.0.

Hydrophobic interaction chromatography (HIC)—The refolded solution was subjected to hydrophobic interaction chromatography. The conductivity of the solution was increased by addition of sodium chloride and then protein was loaded onto hydrophobic interaction resin. The proinsulin was eluted with the increasing gradient of sodium chloride in glycine buffer.

Enzymatic cleavage by trypsin—The protein eluted from HIC was digested with 1:5000 ratio of protein to trypsin. The reaction was carried out at 4° C. and pH 11.2. The reaction was monitored by HPLC analysis. After complete digestion, reaction was quenched by addition of acetic acid.

Cation exchange chromatography—The digested protein was further purified by cation exchange chromatography. The protein was loaded onto cation exchange chromatography and eluted with buffer containing

Sodium Chloride. The Insulin glargine was eluted by using increasing gradient of Sodium Chloride.

Reverse phase chromatography—The active insulin is purified from digested sample by reverse phase chromatography. The protein is loaded to achieve final binding in the range of 10-15 mg/ml of resin. The insulin is eluted using increasing gradient of acetonitrile.

### Example 11: Comparison of Expression Level and Yield of Insulin and Insulin Analogues Using Different Leader Peptides

TABLE 1
Percent expression levels in the absence and presence
of leader peptides of the present invention

| | Expression level (% of total cellular protein) | | |
|---|---|---|---|
| | Without | SEQ ID | SEQ ID |
| Insulin analogue | leader | NO: 1 | NO: 2 |
| Human Insulin | ~8% | ~25% | ~40% |
| Human Insulin Lispro | ~8% | ~25% | ~45% |
| Human Insulin Glargine | ~6% | ~25% | ~40% |

The expression of Insulin or its analogues was considerably less without leader peptide as compared to the expression in the presence of a leader peptide.

TABLE 2
Final yield of the protein in presence of
leader peptides of the present invention

Final yield (g/L)

| | SEQ ID | SEQ ID |
|---|---|---|
| Insulin analogue | NO: 1 | NO: 2 |
| Human Insulin | 0.16-0.24 | 0.4-0.5 |
| Human Insulin Lispro | 0.16-0.2 | 0.4-0.5 |
| Human Insulin Glargine | 0.08-0.1 | 0.16-0.18 |

As observed, the presence of the leader peptide sequences of the present invention enhanced the expression of insulin and insulin analogues and the final yield of the protein of interest.

From the foregoing, it will be appreciated that, although specific embodiments of the invention have been described herein merely for purposes of illustration, various modifications may be made without deviating from the spirit and scope of the invention and should not be construed so as to limit the scope of the invention or the appended claims in any way

1. A process for producing insulin and insulin analogues comprising pre-proinsulin of Formula 1:
R₁—X₁-X₂-X₃ Formula 1
as an intermediate, wherein X₁ is a ‘B’ chain of insulin or insulin analogues, X₂ is a dipeptide selected from RR or KR or RK or KK, X₃ is an ‘A’ chain of insulin or insulin analogues and R₁ is a leader peptide sequence selected from
a) a peptide having amino acid sequence as set forth of in SEQ ID NO: 2 or
b) a peptide having at least 80% homology to a).

2. The process as claimed in claim 1, wherein the leader peptide having at least 80% homology is selected from:
a) a peptide having amino acid sequence as set forth in SEQ ID NO: 1,
b) a peptide comprising amino acid sequence of: MSRIVINAYAKATQP;
c) a peptide comprising amino acid sequence of: MEKHTKDQIIEAPHM.

3. The process as claimed in claim 1, wherein the process comprises preparation of proinsulin of Formula 2: X₁-X₂-X₃ from pre-proinsulin, wherein X₁ is a ‘B’ chain of insulin or insulin analogues, X₂ is a dipeptide selected from RR or KR or RK or KK, and X₃ is an ‘A’ chain of insulin or insulin analogues.

4. The process as claimed in claim 3, wherein the process comprises expression of proinsulin by culturing prokaryotic host cells comprising a nucleic acid encoding proinsulin operably linked to the leader peptide in a production medium.

5. The process as claimed in claim 4, wherein the prokaryotic host cell is selected from Pseudomonas cell or Escherichia coli cell.

6. (canceled)

7. A polypeptide comprising a leader peptide operably linked to the precursor of insulin or insulin analogues, wherein the leader peptide is selected from:
a) a peptide having amino acid sequence as set forth in SEQ ID NO: 2,
b) a peptide having at least 80% homology to a).

8. (canceled)

9. (canceled)

10. (canceled)

11. A leader peptide sequence selected from:
a) a peptide having amino acid sequence as set forth in SEQ ID NO: 2,
b) a peptide having at least 80% homology to a).

12. (canceled)

13. (canceled)

14. (canceled)

15. A nucleotide sequence encoding amino acid sequence of Formula 1, R₁—X₁-X₂-X₃, wherein X₁ is a ‘B’ chain of insulin or insulin analogues, X₂ is a dipeptide selected RR or KR or RK or KK, X₃ is an ‘A’ chain of insulin or insulin analogues and R1 is selected from:
a) a peptide having amino acid sequence as set forth in SEQ ID NO: 2,
b) a peptide having at least 80% homology to a).

16. The nucleotide sequence as claimed in claim 15, wherein the sequence is selected from SEQ ID NO: 11, SEQ ID NO: 12, SEQ ID NO: 13, SEQ ID NO: 14, SEQ ID NO: 15, and SEQ ID NO: 16.

17. A recombinant gene construct comprising nucleotide sequence as claimed in claim 15.

18. The recombinant gene construct as claimed in claim 17, wherein the gene construct is selected from pET28aULL1INS or pET28aULL2INS, for expression of Insulin, pET28aULL1LSP or pET28aULL2LSP for expression of Insulin Lispro, and pET28aULL1GR or pET28aULL2GR for expression of Insulin Glargine.

19. (canceled)

20. (canceled)

21. (canceled)

22. (canceled)

23. (canceled)

24. (canceled)

25. (canceled)

26. Insulin or Insulin analogues obtained by the process as claimed in claim 1, comprising pre-proinsulin of Formula 1:
R₁—X₁-X₂-X₃ Formula 1
as an intermediate, wherein X₁ is a ‘B’ chain of insulin or insulin analogues, X₂ is a dipeptide selected RR or KR or RK or KK, X₃ is an ‘A’ chain of insulin or insulin analogues and R₁ is the leader peptide selected from
a) a peptide having amino acid sequence as set forth of in SEQ ID NO: 2 or
b) a peptide having at least 80% homology to a).

27. The Insulin or Insulin analogues as claimed in claim 26, wherein the leader peptide having at least 80% homology is selected from:
a) a peptide having amino acid sequence as set forth in SEQ ID NO: 1,
b) a peptide comprising amino acid sequence of: MSRIVINAYAKATQP;
c) a peptide comprising amino acid sequence of: MEKHTKDQIIEAPHM.

28. The leader peptide as claimed in claim 11, wherein the leader peptide having at least 80% homology is selected from:
a) a peptide having amino acid sequence as set forth in SEQ ID NO: 1,
b) a peptide comprising amino acid sequence of: MSRIVINAYAKATQP;
c) a peptide comprising amino acid sequence of: MEKHTKDQIIEAPHM.
